(19) United States
(12) Patent Application Publication
(10) Pub. No.: US 2024/0108780 A1
(43) Pub. Date: Apr. 4, 2024

(54) AIRBORNE PATHOGEN DESTRUCTION SYSTEM

(71) Applicant: BATTELLE SAVANNAH RIVER ALLIANCE, LLC, Aiken, SC (US)

(72) Inventors: ELIEL VILLA-ALEMAN, AIKEN, SC (US); JOHN T. BOBBITT, III, EVANS, GA (US); WENDY KUHNE, EVANS, SC (US); MICHAEL C. MAXWELL, AIKEN, SC (US); MATTHEW FOLSOM, AIKEN, SC (US); CHARLES TURICK, AIKEN, SC (US); COURTNEY BURCKHALTER, TRENTON, SC (US); TRAVIS HUBBARD, WARRENVILLE, SC (US); TYLER BARNES, CORDOVA, SC (US); JOSEPH KINNEY, AIKEN, SC (US)

(21) Appl. No.: 17/955,882

(22) Filed: Sep. 29, 2022

(51) Int. Cl. A61L 9/22; A61L 9/03

(52) U.S. Cl. CPC A61L 9/22; A61L 9/032; A61L 2209/14

(57) ABSTRACT

Air purification devices are described. Devices are powered to form an electric field along an airflow path that creates a plasma for destruction of pathogens carried through the device. Devices can be single-user, portable devices for purification of air flow to and/or from a single user.

### FEDERAL RESEARCH STATEMENT

This invention was made with Government support under Contract No. DE-AC09-08DR22470, awarded by the U.S. Department of Energy. The Government has certain rights in the invention.

### BACKGROUND

Typical air purifiers work by removing particulates from the air via filtration or electrostatic collection. Electrostatic-type devices utilize an electric field to collect and trap particulates, including pathogenic particulates (bacterial, viral, etc.), in/on a collection device retained within the purifier. In some devices, a disinfection agent, e.g., ultraviolet (UV) energy, ozone, hydrogen peroxide, or the like is utilized in conjunction with the collection system. Unfortunately, such disinfection agents are slow acting unless extremely high (toxic) concentrations of the agents are utilized. For instance, there exist sterilizers designed to disinfect an entire room by emission of very powerful UV pulses to destroy any pathogens contacted by the UV energy. However the required energy level is so high that people cannot be present during operation of the device. Even with such high power UV, a disinfection cycle takes about five minutes and at least two cycles are required to sterilize an entire room.

Pathogens are not destroyed as they are carried through existing collection-type devices. As such, pathogens that evade capture and collection exit the device without loss of pathogenic function and pathogens that are captured are still active in/on the collection device. As the devices can also retain moisture from the passing air, the pathogens held by the collection device can retain activity for a period of time and even continue to develop and multiply, turning the collection device into a pathogenic breeding ground. The addition of a disinfectant agent (UV, ozone, etc.) to the device is often used to destroy pathogens that that have been retained by the device, but due to low disinfectant activity (due to non-toxic levels and resulting long exposure times necessary for disinfection), pathogens that pass through the device without capture are not likewise destroyed and pathogens that multiply prior to disinfection by the agent can infect other areas of the device.

What are needed in the art are air purification devices capable of destroying pathogens that pass through the device, irrespective of capture of the pathogens. Portable, single-user devices that can destroy airborne pathogens and disinfect the inhaled and/or exhaled air of a user could be of great benefit in the art.

### SUMMARY

According to one embodiment, disclosed is an airborne pathogen destruction device. A device can include an airflow path having a largest cross sectional dimension of about 1 inch or less and configured in some embodiments to carry air at a relatively low flow rate, e.g., about 10 liters per minute (L/min) or less, along the path, though devices can also be designed to carry air at much higher flow rates, if desired. The device can also include a power source and first and second electrodes in electrical communication with the power source. The first electrode can extend along a length of the path and the second electrode can be located at a distance from the first electrode such that an electric field can be established along the length of the path and between the first and second electrodes. The power source can be configured to establish an electric field of about 6 kilovolts (kV) or greater along the path length. A device can optionally include a filter or the like for removal of materials carried in the airflow. In some embodiments, a device can be a personal air purification device designed to purify air of a single user as it is inhaled and/or exhaled.

Also disclosed is a method for destroying an airborne pathogen. The method can include passing air along an airflow path of a device as described. Due to the high voltage of the electric field that extends along the path and contact time between the air and the electric field due to a low flow rate, e.g., about 10 L/min or less, the air can become ionized and form a plasma field along the length and pathogens present in the air can be destroyed as they pass through the field. As the ionization of the air can create undesirable materials, e.g., ozone and devitalized pathogen debris, the method can optionally include removal of such components from the flow prior to exit from the device.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates an airflow path of a device as disclosed herein.

FIG. 2 schematically illustrates a portable device as disclosed herein.

FIG. 3 compares the collected debris of airflow samples following passage through an electric field of a device and the collected debris of airflow samples following passage through the same device with no electric field.

FIG. 4 illustrates an experimental set-up incorporating a device as disclosed herein.

FIG. 5 compares scanning electron microscope (SEM) images of control samples and test samples containing E. coli. collected following passage through a device as described

FIG. 6A illustrates the results of a colony formation assay for samples exposed to a negative polarity system including a 0.625 inch (1.59 cm) diameter flow path.

FIG. 6B illustrates the results of a colony formation assay for samples exposed to a negative polarity system including a 0.5 inch (1.27 cm) diameter flow path.

FIG. 7 presents electrophoresis data demonstrating the degradation of pathogen RNA by disclosed systems.

FIG. 8 presents quantitative reverse transcription PCR (RT-qPCR) of essential genes demonstrating the degradation of pathogen RNA by disclosed systems.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to air purification devices that can destroy airborne pathogens that pass through the devices. More specifically, disclosed devices include a power source and associated electronics designed to create an electric field along an airflow path of the device. Upon passage of air through the electric field, the air becomes ionized, creating a plasma field. Pathogens, including bioaerosols such as bacteria, virus, spore formers, and the like, carried in the air can be destroyed as they pass through the plasma field. While not wishing to be bound to any particular theory, it is understood that the electric field itself as well as ions of the plasma field contribute to the destruction of pathogens. In some embodiments, the characteristics of the electric field can be tuned such that UV (e.g., UVC) and/or ozone are generated in the plasma, both of which can additionally contribute to the destruction of pathogens carried in the field.

Beneficially, as airborne pathogens can be destroyed as they pass through the electric field within the device, there is no need to incorporate separate sources of disinfection agents. Moreover, as pathogens are rendered harmless upon passage through the device, disclosed devices need not include large, complicated particle removal components, such as filters, collection plates or grids. As such, disclosed devices can be simpler and require less cleaning and part replacement than other, previously known collection-type devices. Moreover, the lack of such internal components can decrease the chance of living pathogens collecting within the device, where they could propagate and create a new biohazard that can create infection risk when cleaning or replacing such components. Of course, and as discussed further herein, in some embodiments, a device can include an added filtration or other device for removal of materials from the outflow of the device.

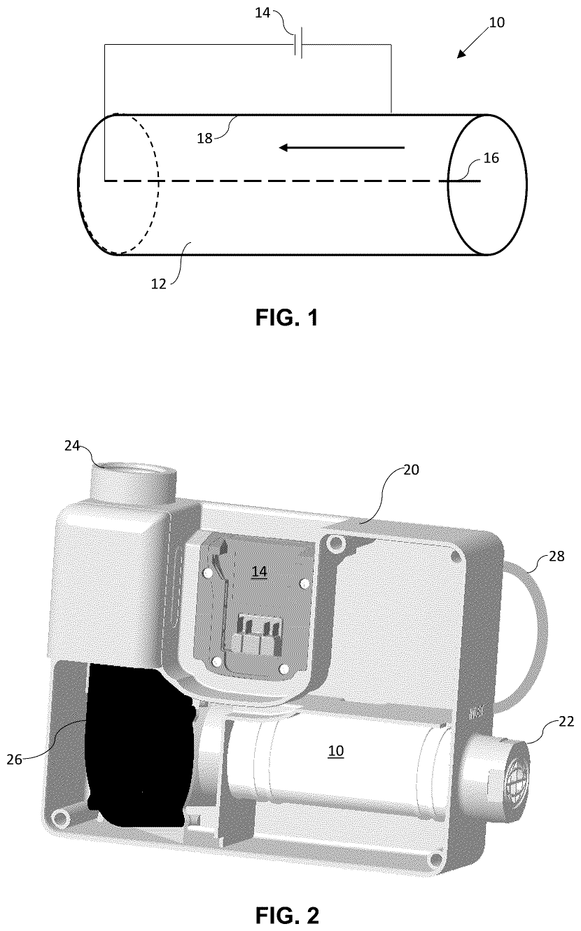

FIG. 1 schematically illustrates one embodiment of pathogen destruction segment 10 of a device. The segment 10 includes an airflow path 12 through which air can flow as indicated by the directional arrow. The segment 10 includes a power source 14 in electrical communication with first and second electrodes 16, 18 that can establish an electric field in the airflow path 12.

The electrical system combined with the geometry of the segment 10 can be designed to produce an electric field that extends along the length of the segment 10. To ensure that the electric field across the width of the entire flow path is strong enough to render pathogens in a passing airflow harmless the airflow path 12 can be relatively narrow in some embodiments, which can also allow for small size (e.g., portability) and ease of use of a device. For instance, an airflow path 12 can have a largest cross-sectional dimension (e.g., diameter) of about 1 inch (2.5 cm) or less, such as about 0.75 inches (2 cm) or less, about 0.6 inches (1.6 cm) or less, or about 0.5 inches (1.3 cm) or less in some embodiments, such as from about 0.25 inches (0.6 cm) to about 1 inch (2.5 cm) in some embodiments.

The airflow path 12 can also be designed to prevent capture of pathogens or pathogenic debris along the path. For instance, the airflow path 12 can be designed with as few turns and corners as possible, e.g., a straight path with a circular or ovoid cross section that includes no corners that could trap pathogens or pathogenic debris.

In one embodiment, the power source 14 can be a battery, either single-use or rechargeable, that can allow for untethered use of a device. In such embodiments, a user can carry a portable device for continuous availability or use. Any battery as known in the art can be utilized to provide suitable power to establish an electric field along the airflow path 12. For instance, a rechargeable lithium-ion battery or the like that can provide on-board recharging, or a single-use replaceable battery could be utilized as a suitable power source. In other embodiments, the power source can require tethering, e.g., connection to an outlet or the like.

The power source 14 is in electrical communication with electrodes 16, 18. In the illustrated embodiment of FIG. 1, one of the electrodes 16 can be located within the airflow path 12, for instance axially located within the center of the airflow path. In such an embodiment, the electrode 16 can be designed so as to not interfere with airflow. For instance, the electrode 16 can be an electrically conductive wire, e.g., a corona wire such as a corona wire composed of titanium, stainless steel, nichrome, or the like.

The second electrode 18 can be at an outer edge of the airflow path 12 so as to establish an electric field between the two electrodes 16, 18. For instance, in the illustrated embodiment, the second electrode 18 can be at the surface of the enclosed airflow path 12. As such, the second electrode 18 can include an inner surface of the wall enclosing the airflow path 12, e.g., an electrically conductive surface of stainless steel or the like. Such a surface can be a coating on the wall or can form the entire wall of the airflow path 12 provided, of course, that the surface will be electrically insulated from surrounding components of a device as well as isolated from a user.

Beneficially, embodiments in which the first electrode 16 is in the form of a single, central wire along the airflow path 12 and the second electrode 18 surrounds the first electrode, the system can utilize a single wire as the first electrode 16, which can prevent interruption of flow through the device as well as establish a well-defined electric field along the length of the airflow path 12.

The length and strength of the electric field established along the airflow path 12 can be such to ensure destruction of pathogens carried along the path. For instance, the system can establish a static electric field along the flow path of about 1 kV or greater, such as about 2 kV or greater, about 3 kV or greater, about 4 kV or greater, about 5 kV or greater, about 6 kV or greater, such as about 6.5 kV or greater, about 7 kV or greater, about 8 kV or greater, about 8.5 kV or greater, or about 9 kV or greater in some embodiments, such as from about 1 kV to about 10 kV, from about 2 kV to about 10 kV, or from about 3 kV to about 9 kV in some embodiments.

The path length of the electric field can be varied depending upon the voltage strength of the electric field as well as other aspects of the device including, without limitation, the overall size of the device, the flow rate expected through the airflow path 12, the expected maximum pathogen load, etc. In general, however, the design of the electrodes can be such to establish an electric field along an airflow path 12 for a distance of about 1 inch (2.5 cm) or greater, about 1.5 inches (3.8 cm) or greater, or about 2 inches (5 cm) or greater in some embodiments, such as about 5 inches (13 cm) or less, or about 4 inches (10 cm) or less in some embodiments. By way of example an electric field can be established along a length of an airflow path 12 of from about 1.5 inches (3.8 cm) to about 4.5 inches (11 cm) or from about 2 inches (5 cm) to about 4 inches (10 cm) in some embodiments. In some embodiments, the length of the electric field can be absent of any turns or corners.

The pathogen destruction segment 10 can be a component of a device 20 such as illustrated in FIG. 2. As illustrated, a device 20 can include an inlet 22 through which an airflow input can be provided and an outlet 24 through which processed air can exit the device. As discussed above, the device need not include any pre-filtration or collection devices (e.g., electrostatic collection plates, filters, etc.) as are common in previously known systems. A power supply 14 (or connector to a power supply) can supply power to the segment 10 and establish an electric field therein. Upon entry of air to the segment 10, the electric field can ionize the air. The ions and electrons of the field can interact with pathogens present in the air flow. While complete understanding to the destructive aspects of the passage are not fully known, it is understood that destruction can occur via damage to cell membrane, protein coats, etc. as well as degradation of nucleic acids (DNA, RNA) that leads to devitalization and loss of pathogenic capabilities of the pathogens.

A device is not limited to a single segment 10, and in other embodiments, a device can include multiple airflow paths. For instance, a device can include a single air intake with the intake path then split to deliver air to multiple segments 10, each of which provides for the pathogen destruction therein. Such devices can be designed to purify a large volume of air, such as a room purification device.

As discussed previously, due to the destruction of pathogens carried through disclosed systems, separation (filtration, electrostatic collection, etc.) is not necessary either prior to air passing through the destruction segment 10 or following exit from the segment 10. However, in some embodiments, additional components can be included in a device to further purify an exit stream. For instance, ozone can be a by-product generated within the destruction segment 10. The residence time of pathogens within the created ozone will be relatively short and as such, it may contribute to destruction of pathogens, but will not have a great impact on the effectiveness of the devices as it will be relatively inconsequential to the destruction as compared to the effect of the plasma. As ozone can be an irritant to humans at relatively low levels (e.g., about 0.8 ppm), a device can include a filter 26, e.g., an activated carbon filter, that can remove ozone and optionally other materials (e.g., particulates) from an air flow.

When present, filter 26 can be replaceable and/or rechargeable. For example, in one embodiment filter 26 can be replaceable in conjunction with a replaceable power supply 14. In one embodiment, a filter 26 can be a rechargeable filter that can be cleaned, e.g., purged of ozone and other collected materials, for reuse. For instance, a filter 26 can be removed, cleaned, and replaced into the device 20 for additional use.

During operation, flow through the device can be active or passive. For instance, a device can include an in-line fan or a top fan, for instance a fan can be located within a device in conjunction with a filter 26 or any other convenient location. A fan can pull or push air through the pathogen destruction segment. When included, a fan can provide airflow through the device at a relatively low air speed, so as to ensure suitable contact time of pathogens contained within the airflow with the plasma of the destruction segment 10. In other embodiments, it may be preferred to not include any active air flow mechanisms, for instance air flow through a device can be enabled purely by the respiration of a user. Air flow through a device can be selected depending upon intended use. For instance, when considering utilization of a device for a single-user, i.e., purifying only air inhaled and/or exhaled from a single user, a fan can provide airflow through a destruction segment at a relatively low rate, such as about 10 L/min or less, such as about 8 L/min or less or about 5 L/min or less, for instance from about 2 L/min to about 10 L/min in some embodiments. In other applications, a device can be designed for higher air flow rates, such as 100 L/min, or even more in some embodiments.

Optionally, a fan providing forced airflow through a device can be a variable speed fan, to allow adaptation of a device depending upon different operating conditions.

A device can include one or more sensors and notification components that can monitor characteristics of a device such as, and without limitation to, loss of electric field at the destruction segment 10, the amount of ozone in an exit flow, low battery condition, inadequate or excessive airflow through a device, and the like. Notification components can be on-board systems utilizing visual, auditory, or kinesthetic notifications and/or remote systems, e.g., blue-tooth, Wi-Fi, cellular, or other types of remote communications systems that can provide information regarding the characteristics of a device. Such systems can be powered by the power supply 14 that provides power to the pathogen destructions segment 10 or by use of a separate power supply. In some embodiments, a device can include a secondary backup battery that can provide for notification if the primary power supply fails as well as maintain function of the device during replacement/charging of the primary power supply 14.

In one embodiment, a device can be a single-user device designed to be carried by and utilized by an individual. As such, a device can include one or more components to allow a user to carry or wear a device. For example, a device can include one or more connectors 28, e.g., hooks, straps, clamps, or the like that can allow a user to connect a device to a belt, clothes, lanyard, etc. for truly portable use.

Disclosed devices can be utilized to provide air free of pathogens to an individual as well as to decontaminate pathogen-containing breath from an individual. For instance, a device can be utilized in conjunction with a ventilator or oxygen mask to decontaminate exhausted air from a patient, and in such an embodiment can prevent contamination of equipment and materials present in the surrounding area such as lab coats, gloves, masks, etc. Likewise, a device can be utilized to provide decontaminated air to a user, e.g., to decontaminate air prior to inhalation so as to prevent infection by airborne pathogens for a wearer in public, in a high-risk medical environment, or any other location. In such an embodiment, the decontaminated air that exits the device can be supplied to a hood worn by a user, a mask, supply air to a ventilator, or simply to the area in which the user is located, e.g., to the local room.

To provide for wide variability in use, the inlet 22 and/or outlet 24 can be designed for connection to other components. For example, inlet 22 and/or outlet 24 can be threaded to allow connection to commercially available breathing masks, hoods, face shields, ventilators, and/or other personal protective equipment.

The present invention may be better understood with reference to the examples, set forth below.

### Example 1

A system as described herein was examined for effect on an air sample that was loaded with E. coli. The system was run with power to the flow path, i.e., establishing an electric field in the flow path, as well as with no power to the flow path as a control. Debris was collected from air as it exited the device.

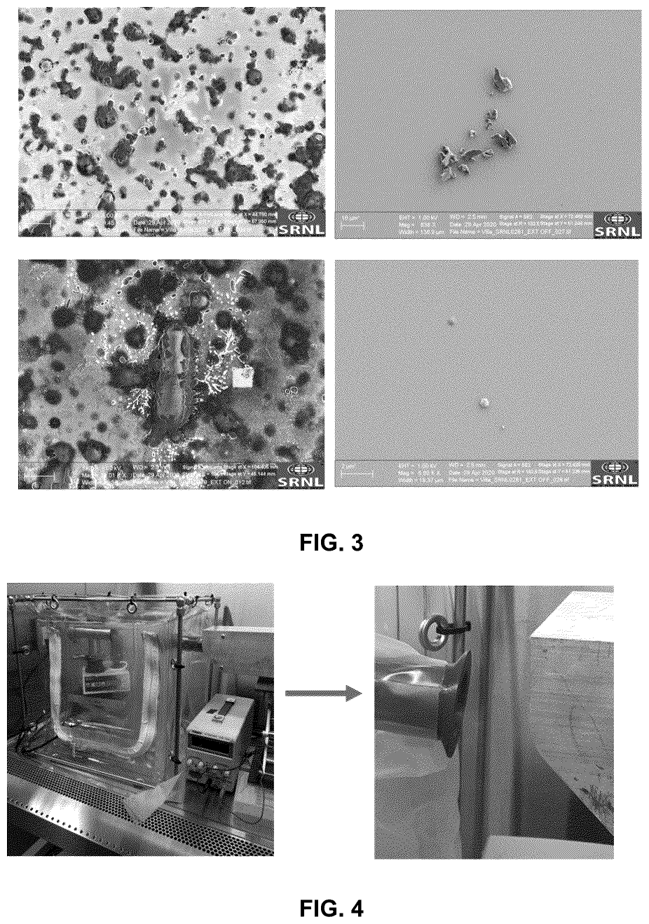

Results are shown in FIG. 3 including images of the debris field for the system when the electric field was established in the flow path (left) and images of the debris field with no electric field in the flow path (right). The images of the debris field are characteristic of carbon, believed to be the result of the outer and inner membranes of the E. coli rupturing and depositing the inner cytoplasm and nuclear DNA onto the collection plate. With no electric field in the flow path, essentially no debris was collected. The rod-shaped structure in the lower left panel (about 1.5×0.5 micrometers) is believed to be a captured, intact E. coli.

### Example 2

A “cloud” of aerosol containing E. coli was generated inside a glove bag (FIG. 4, left) and a fan was used to evacuate the bag for about 3 minutes and deliver the aerosol to a device as described herein (FIG. 4, right). A tryptic soy agar plate was held at the exhaust of the device for about 20 seconds to collect material exhausted from the system. E. coli was maintained in DI water to reduce the amount of salt entering the device and deposited onto the collection plates.

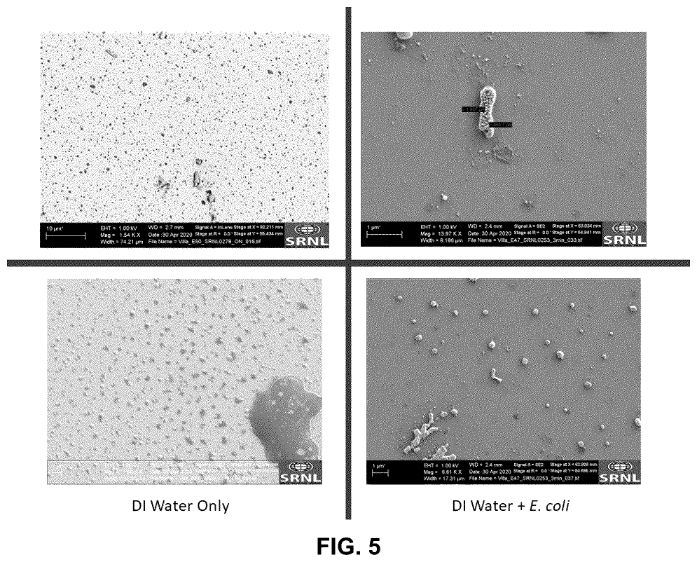

Plates obtained from samples run with DI water only as control and samples including DI water with E. coli were submitted for SEM analysis. Results are shown in FIG. 5 with control images on the left and E. coli containing samples on the right. On the DI water only plates (left) very distinctive water droplets are seen and some carbon material that might be from the plastic bags in which the plates were shipped. On the plates containing E. coli (right) a greater field of “debris” was observed with what appears to be a few intact E. coli. The observance of intact E. coli is not out of question given the reduced current at which the device was run.

### Example 3

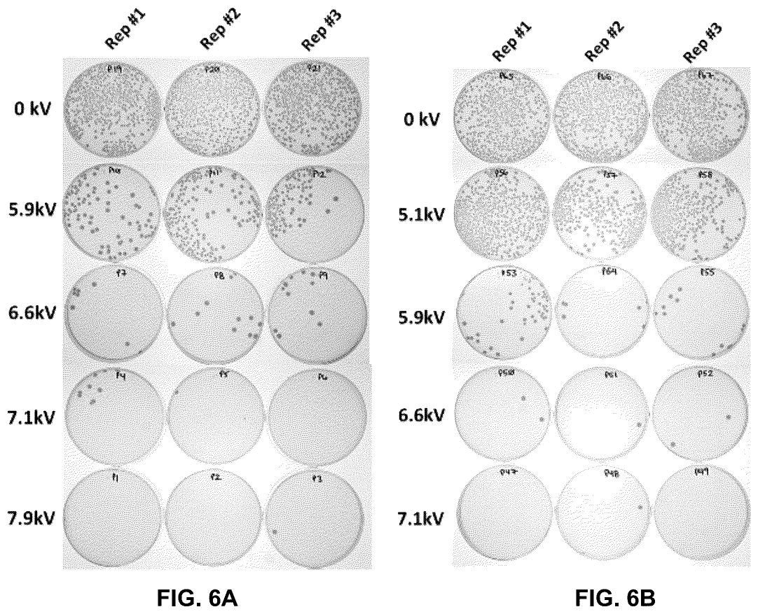

Two different systems including airflow path diameters of 0.625 inch (1.59 cm) and 0.5 inch (1.27 cm) with negative polarity were run with a variety of different voltages established in the flow path. Results are shown in FIG. 6A for the larger flow path diameter and in FIG. 6B for the smaller flow path diameter. As can be seen, effective reduction of live E. coli was obtained for both systems at voltages of 6.6 and above, with the smaller diameter flow path device demonstrating effective E. coli reduction at lower voltages.

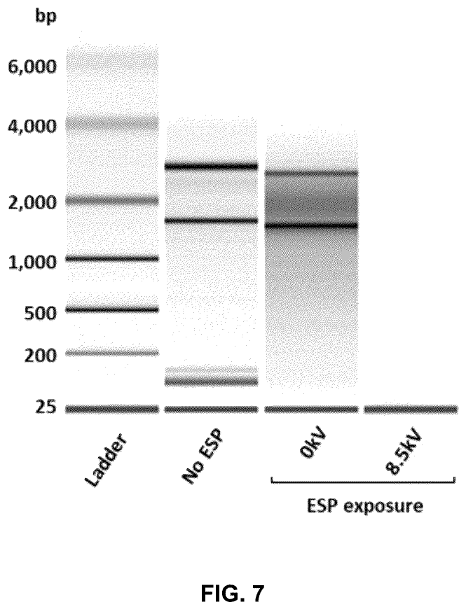

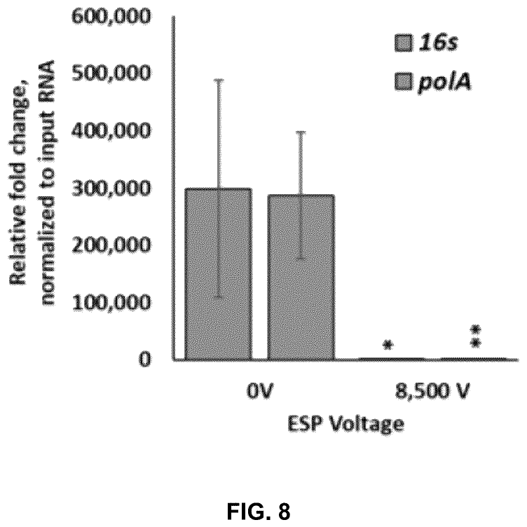

Damage to genomic material was also investigated by evaluating the RNA integrity of the collected E. coli by electrophoresis (FIG. 7) and RT-qPCR (FIG. 8). RNA integrity was minimally affected after exposure to the field at 0 kV while exposure to 8.5 kV caused complete RNA degradation, as indicated by the loss of prokaryotic ribosomal RNA, 16 s and 23 s. Upon RT-qPCR investigation of mRNA, it was determined that E. coli gene expression of two critical self-replication genes, 16 s and polA, was abolished (FIG. 7, FIG. 8). Together, these results indicate that after exposure to the system at 8.5 kV, RNA was degraded and no longer detectable, preventing any future self-replication and indicating complete disinfection of the bacteria.

While certain embodiments of the disclosed subject matter have been described using specific terms, such description is for illustrative purposes only, and it is to be understood that changes and variations may be made without departing from the spirit or scope of the subject matter.

What is claimed is:

1. An airborne pathogen destruction device comprising:
an airflow path;
a power source;
a first electrode in electrical communication with the power source, the first electrode extending along a length of the airflow path; and
a second electrode in electrical communication with the power source, the second electrode extending along the length of the airflow path; wherein
the power source is configured to establish an electric field of about 6 kilovolts or greater between the first electrode and the second electrode and along the length of the airflow path.

2. The airborne pathogen destruction device of claim 1, wherein the first electrode comprises a single corona wire.

3. The airborne pathogen destruction device of claim 1, wherein the length of the airflow path is surrounded by a wall, the surrounding wall comprising a largest cross-sectional dimension of about 1 inch or less.

4. The airborne pathogen destruction device of claim 3, wherein the second electrode comprises a surface of the wall.

5. The airborne pathogen destruction device of claim 1, further comprising a filter in fluid communication with the airflow path.

6. The airborne pathogen destruction device of claim 5, wherein the filter comprises activated carbon.

7. The airborne pathogen destruction device of claim 1, wherein the length of the airflow path is a straight path free of corners and turns.

8. The airborne pathogen destruction device of claim 1, wherein the power supply comprises a rechargeable battery.

9. The airborne pathogen destruction device of claim 1, wherein the length is from about 1 inch to about 5 inches.

10. The airborne pathogen destruction device of claim 1, further comprising a fan configured to pull or push air along the airflow path.

11. The airborne pathogen destruction device of claim 1, further comprising one or more sensors for detection of ozone, an electronic condition, or an airflow condition within the device.

12. The airborne pathogen destruction device of claim 1, wherein the device is a single-user device.

13. The airborne pathogen destruction device of claim 12, the device further comprising a connector configured for attachment of the device to a user.

14. The airborne pathogen destruction device of claim 12, wherein the device is connectable to a breathing mask, a hood, a face shield, or a ventilator.

15. A method for destroying an airborne pathogen, comprising passing air along an airflow path within a device at a rate of about 10 liters per minute or less, the airflow path comprising an electric field along a length of the path, the electric field having a voltage of about 6 kilovolts or greater, the air becoming ionized and forming a plasma field along the length, wherein pathogens within the air are destroyed upon passing through the plasma field.

16. The method of claim 15, wherein nucleic acids of the pathogens are degraded upon passing through the plasma field.

17. The method of claim 15, further comprising modifying the flow rate of air through the airflow path.

18. The method of claim 15, further comprising monitoring one or more of an ozone level, a battery condition, or the flow rate in the device.

19. The method of claim 15, further comprising attaching the device to a breathing mask, a hood, a face shield, or a ventilator.

20. The method of claim 15, wherein the air that is passed along the airflow path originates from or is delivered to a single user.
